(19) United States
(12) Patent Application Publication
(10) Pub. No.: US 2009/0209754 A1
(43) Pub. Date: Aug. 20, 2009

(54) Process for the preparation of capecitabine

(76) Inventors: Peter Lindsay MacDonald, Gentilino (CH); Pierluigi Rossetto, Lodi (IT); Maurizio Gallina, Novara (IT)

Correspondence Address: KENYON & KENYON LLP, ONE BROADWAY, NEW YORK, NY, 10004, US

(21) Appl. No.: 12/319,292

(22) Filed: Jan. 5, 2009

Related U.S. Application Data
(60) Provisional application No. 61/018,802, filed on Jan. 3, 2008.
(60) Provisional application No. 61/067,608, filed on Feb. 28, 2008.
(60) Provisional application No. 61/127,851, filed on May 15, 2008.
(60) Provisional application No. 61/058,750, filed on Jun. 4, 2008.
(60) Provisional application No. 61/061,861, filed on Jun. 16, 2008.
(60) Provisional application No. 61/079,306, filed on Jul. 9, 2008.
(60) Provisional application No. 61/107,818, filed on Oct. 23, 2008.
(60) Provisional application No. 61/109,693, filed on Oct. 30, 2008.

(51) Int. Cl. C07H 19/06

(52) U.S. Cl. 536/28.51

(57) ABSTRACT

The present application relates to an improved process for the preparation of capecitabine.

### CROSS-REFERENCE TO RELATED APPLICATIONS

This application claims the benefit of U.S. Provisional Application Nos. 61/018,802, filed Jan. 3, 2008; 61/067,608, filed Feb. 28, 2008; 61/127,851, filed May 15, 2008; 61/058,750, filed Jun. 4, 2008; 61/061,861, filed Jun. 16, 2008; 61/079,306, filed Jul. 9, 2008; 61/107,818, filed Oct. 23, 2008; and 61/109,693, filed Oct. 30, 2008, hereby incorporated by reference.

### FIELD OF INVENTION

The present application relates to an improved process for the preparation of capecitabine.

### BACKGROUND OF THE INVENTION

Capecitabine, 5′-deoxy-5-fluoro-[N⁴-(pentyloxy) carbonyl]-cytidine, a compound having the following chemical structure,

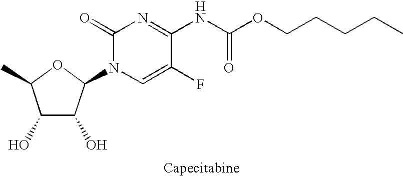

is a fluoropyrimidine carbamate with antineoplastic activity. Capecitabine is marketed under the trade name Xeloda® by Roche. It is an orally administered systemic prodrug of 5′-deoxy-5-fluorouridine (5′-DFUR), which is converted to 5-fluorouracil. It is indicated as a single agent for adjuvant treatment in patients with Dukes' C colon cancer and metastatic colorectal carcinoma.

The synthesis of capecitabine is described in several publications, U.S. Pat. Nos. 5,472,949 (“'949 patent”), 4,966,891 (“'891 patent”), 5,453,497 (“'497 patent”) and 5,476,932 (“'932 patent”). The processes can be summarized by the following scheme:

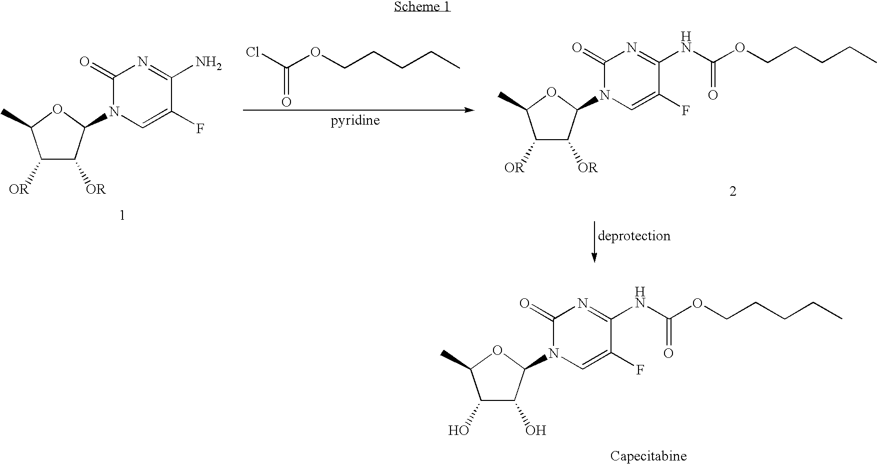

wherein R is C(O)CH₃ in the '949 and the '497 patents, R is SiMe₃ in the 891' patent, and R is C(O)C₅H₁₁ in the '932 patent. In these patents, the compound of formula 1 is acylated by using excess of pyridine and acylating agent, which is undesirable both for economical and environmental reasons. Furthermore, the compound of formula 2 is recovered before it is converted to capecitabine, where according to the '949 and the '497 patents, the recovery includes distilling the excess of pyridine, an operation which is undesirable due to safety reasons. Then, the recovered compound of formula 2 is reacted at a temperature between 0° C. and 30° C. with aqueous sodium hydroxide in the presence of methanol, providing capecitabine. Capecitabine is purified either by crystallization from ethyl acetate and heptane as described in the '949 patent, or by column chromatography purification, as described in the '891 patent. As such, column chromatography is a time consuming operation and is not desirable for industrial scale synthesis.

The above processes use excess of pyridine, which is a toxic solvent. Thus, the above processes are not environmental friendly, economic and suitable for industrial scale. Furthermore, using excess of pyridine forces extensive purification, which decreases the product yield.

Therefore, there exists a need for an improved process for preparing capecitabine, which is suitable for industrial scale.

### SUMMARY OF INVENTION

In one embodiment, the present invention encompasses a process for preparing capecitabine of the following formula:

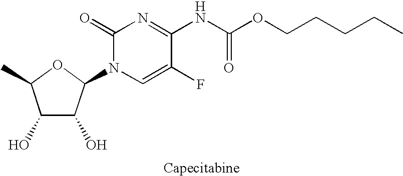

from 2′,3′-di-protected-5′deoxy-5-fluorocytidine (“Pro-5DFC”) of formula 1,

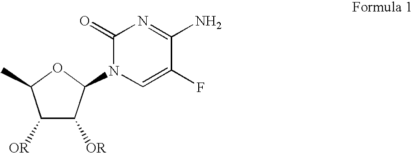

comprising: a) reacting the compound of formula 1 with about 1.1 mole equivalent to about 3.0 mole equivalent of pentyl-haloformate per mole equivalent of the compound of formula 1, and about 1.5 mole equivalent to about 3.2 mole equivalent of a base per mole equivalent of the compound of formula 1 to obtain 2′,3′-di-protected-5′-deoxy-5-fluoro-[N⁴-(n-pentyloxy)carbonyl]-cytidine (“Pro-5DFPCC”) of formula 2; and

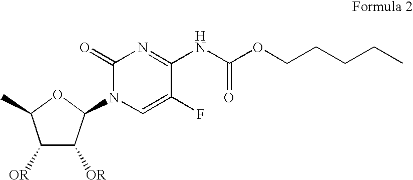

b) removing the protecting groups by hydrolysis at a temperature of about −5° C. to about −25° C. to obtain Capecitabine, wherein R is either C(O)CH₃ or SiMe₃.

In another embodiment, the present invention encompasses a process for preparing Capecitabine from 2′,3′-di-protected-5′-deoxy-5-fluoro-[N⁴-(n-pentyloxy)carbonyl]-cytidine (“Pro-5DFPCC”) of formula 2 comprising removing the protecting groups of compound 2 by hydrolysis at a temperature of about −25° C. to about −5° C. to obtain Capecitabine salt.

In another embodiment, the present invention encompasses a process for preparing 2′,3′-di-protected-5′-deoxy-5-fluoro-[N⁴-(n-pentyloxy)carbonyl]-cytidine (“Pro-5DFPCC”) of formula 2, comprising reacting 2′,3′-di-protected-5′deoxy-5-fluorocytidine of formula 1, about 1.1 mole equivalents to about 3.0 mole equivalents of pentyl-haloformate per mole equivalent of the compound of formula 1 and about 1.5 mole equivalents to about 3.2 mole equivalents of a base per mole equivalent of the compound of formula 1.

In another embodiment, the present invention encompasses a process for preparing Capecitabine comprising preparing 2′,3′-di-protected-5′-deoxy-5-fluoro-[N⁴-(n-pentyloxy)carbonyl]-cytidine (“Pro-5DFPCC”) of formula 2 by the process of the present invention and converting it to Capecitabine.

### DETAILED DESCRIPTION OF THE INVENTION

The present invention relates to an improved process for the preparation of capecitabine in high yield and purity. The processes of the present invention can be illustrated by the following scheme:

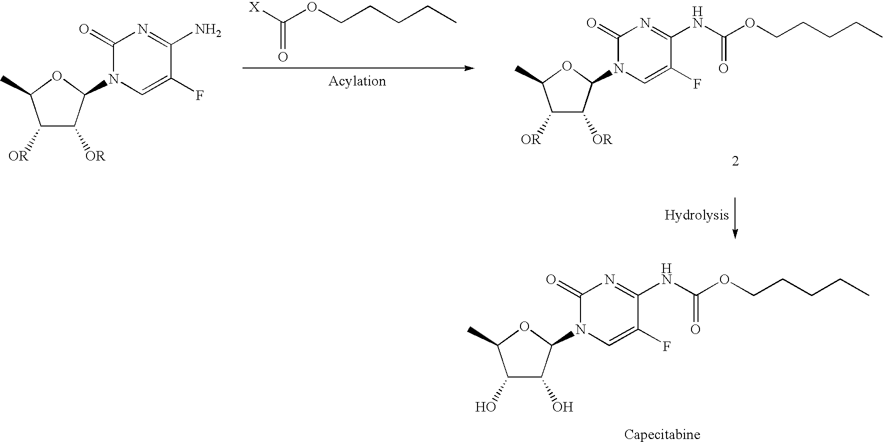

wherein R is either C(O)CH₃ or SiMe₃, and X is a halogen, preferably chlorine.

In this process, the acylation step uses significantly lesser amounts of pyridine and haloformate, and the hydrolysis is done at low temperature, e.g. about −5° C. to about −25° C.

Accordingly, the acylation step of the present invention is more selective, for example, the reaction produces a significantly lesser amount (e.g. less than about 1% to 7% as determined by percentage area HPLC) double acylating (i.e. dipentyl impurity) impurity of the following formula:

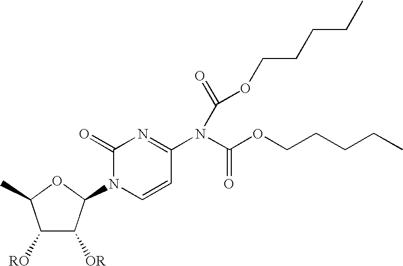

wherein R is either C(O)CH₃ or SiMe₃, which can be formed when excess of haloformate is used.

In addition, the acylation and the hydrolysis can be conducted in a one pot manner, i.e., without the need to isolate the intermediate 2′,3′-di-protected-5′-deoxy-5-fluoro-[N⁴-(n-pentyloxy)carbonyl]-cytidine (“Pro-5DFPCC”) of formula 2. But an organic phase containing it obtained by a simple work up can be used in the hydrolysis step.

Moreover, performing the hydrolysis at low temperatures reduces the formation of 4-amino-1-[(2R,3R,4S,5R)-3,4-dihydroxy-5-methyltetrahydrofuran-2-yl]-5-fluoropyrimidin-2(1H)-one (“impurity A”), an impurity of the following formula:

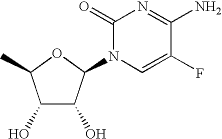

which is obtained by a competing reaction, i.e. further hydrolysis of Capecitabine. Thus, simple purification techniques, such as crystallization, are sufficient to provide highly pure Capecitabine.

When the protecting group of 2′,3′-di-protected-5′deoxy-5-fluorocytidine of formula 1 is O-acetyl (e.g. R═C(O)CH₃), the starting compound 2′,3′-di-O-acetyl-5′deoxy-5-fluorocytidine (“Ac-5DFC”) of the formula 1a:

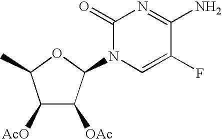

can be prepared, for example by the process disclosed in the '949 patent, hereby incorporated by reference.

When the protecting group of 2′,3′-di-protected-5′deoxy-5-fluorocytidine of formula 1 is O-trimethylsilyl (e.g. R═SiMe₃), the starting compound 2′,3′-di-O-trimethylsilyl-5′deoxy-5-fluorocytidine (“Si-5DFC”) of the formula 1b:

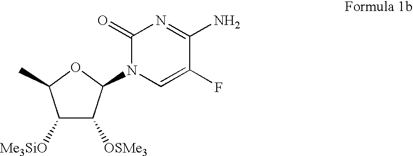

can be prepared by a process comprising reacting 5′deoxy-5-fluorocytidine of formula 3 (“5-DFC”):

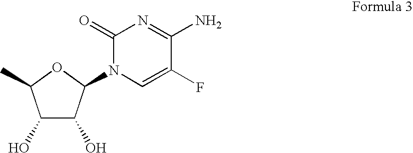

with about 1 mole equivalent to about 5 mole equivalents of trimethylsilylchloride per mole equivalent of the compound of formula 3, as described in examples 11-13.

Typically, the above reaction is done in the presence of a base and a solvent. Preferably, the amount of the base introduced in the protection step is sufficient for both protection step and the proceeding step of acylation. Thus, if the compound of formula 1b (“Si-5DFC”) is not isolated prior to the acylation step no additional base is added to the acylation reaction. Preferably, the amount of base used in the protection and acylation step is about 1.2 mole equivalents to about 5.5 mole equivalents of a base per mole equivalent of the compound of formula 3 (“5-DFC”).

Typically, the base is an organic base or an inorganic base. Preferably, the organic base is pyridine, triethylamine (“TEA”), N,N-Diisopropylethylamine(“DIPEA”), N-methyl-morpholine, imidazole, dimethylaminopyridine(“DMAP”), or mixtures thereof. More preferably, the organic base is pyridine. Preferably, the inorganic base is an alkali metal base or ammonium hydroxide. Preferably, the alkali metal base is sodium carbonate, potassium carbonate, sodium hydrogencarbonate, potassium hydrogencarbonate, magnesium oxide or mixtures thereof. Most preferably, the alkali metal base is potassium carbonate. Most preferably the base is pyridine.

Typically, the protection is done in the presence of a solvent. Preferably, a single solvent or a mixture of solvent is used. Preferably, the solvent is an organic solvent or a mixture of organic solvents. Preferably, the organic solvent is selected from a group consisting of: chlorinated aliphatic hydrocarbons, ketones, esters, ethers, or mixtures thereof. Preferably, the chlorinated aliphatic hydrocarbon is a C₁₋₄ chlorinated aliphatic hydrocarbon, more preferably, dichloromethane. Preferably, the ketone is a C₃-C₆ ketone, more preferably, acetone, methyl-ethyl ketone (“MEK”), methyl-isobutyl ketone (MIBK), or mixtures thereof. Preferably, the ester is a C₄-C₆ ester, more preferably, ethyl acetate, isopropyl acetate, or mixtures thereof. Preferably, the ether is a C₂-C₆ ether, more preferably, C₄-C₆ ether. Most preferably, the ether is 2-methyl-tetrahydrofuran (“2-MeTHF”).

Preferably, the organic solvent is 2-methyl-tetrahydrofuran (“2-MeTHF”). Preferably, when the organic solvent is a mixture, it is a mixture of 2-methyl-tetrahydrofuran (“2-MeTHF”) and at least one of the above solvents.

As mentioned above, the obtained compound of formula 1 (“Pro-5DFC”) can be acylated to give the compound of formula 2 (“Pro-5DFCC”) without being recovered from the reaction mixture of the protection step, i.e., one-pot reaction. Alternatively, the compound of formula 1 is isolated prior to the acylation, thus additional amounts of base and solvent are introduced in the acylation step.

Preferably, the base and the solvent are as described for the protection step. More preferably, the base is pyridine and the solvent is 2-methyl-tetrahydrofuran (“2-MeTHF”).

Preferably, the compound of formula 1 is not isolated prior to the acylation step, and a mixture comprising the compound of formula 1 and a solvent obtained from the protection step, is used for the acylation step.

The said acylation can be achieved by a process comprising: reacting the compound of formula 1 (“Pro-5DFC”) of the following formula:

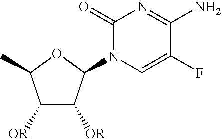

with about 1.1 mole equivalents to about 3.0 mole equivalents of pentyl-haloformate per mole equivalent of the compound of formula 1 and about 1.5 mole equivalents to about 3.2 mole equivalents of base per mole equivalent of the compound of formula 1, to obtain the compound of formula 2 (“Pro-5DFCC”):

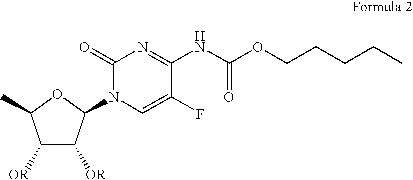

wherein R is either C(O)CH₃ or SiMe₃.

In the above process, the compound of formula 1 (Pro-5DFC) can be used neat (i.e., in the absence of a solvent) or in a mixture with the base and at least one organic solvent. If neat, the compound of formula 1 is preferably combined with an organic solvent, thus providing a solution prior to the addition of the base and the n-pentyl haloformate. Preferably, the organic solvent is as described before. Preferably, the base is as described before.

Preferably, when using the O-acetyl protected compound of formula 1a (“Ac-5DFC”) the amount of pentyl-haloformate is about 1.35 mole equivalents to about 2.0 mole equivalents per mole equivalent of 2′,3′-di-O-acetyl-5′deoxy-5-fluorocytidine of the compound of formula 1a. More preferably, the amount of pentyl-haloformate is about 1.40 mole equivalents to about 1.6 mole equivalents per mole equivalent of the compound of formula 1a.

Preferably, when using the O-trimethylsilyl protected compound of formula 1b (“Si-5DFC”) the amount of pentyl-haloformate is about 1.1 mole equivalents to about 3.0 mole equivalents per mole equivalent of 2′,3′-di-O-acetyl-5′deoxy-5-fluorocytidine of the compound of formula 1b. More preferably, the amount of pentyl-haloformate is about 1.3 mole equivalents to about 3.0 mole equivalents per mole equivalent of the compound of formula 1b.

The haloformate is preferably either chloroformate or bromoformate. More preferably, the haloformate is chloroformate.

Preferably, when using the O-acetyl protected compound of formula 1a (“Ac-5DFC”) the amount of base is about 1.7 mole equivalents to about 2.2 mole equivalents per mole equivalent of the compound of formula 1a. More preferably, the amount of base is about 1.7 mole equivalents per mole equivalent of compound of formula 1a.

As mentioned above, when the compound of formula 1 is not isolated, the same base used for the protection step is used also for the acylation step. Therefore, the amount of the base should be sufficient for the protection and acylation reactions.

Preferably, when the O-trimethylsilyl protected compound of formula 1b (“Si-5DFC”) is isolated, the amount of base is about 1.2 mole equivalents to about 3.2 mole equivalents per mole equivalent of the compound of formula 1b, more preferably about 1.5 mole equivalents to about 3.2 mole equivalents per mole equivalent of the compound of formula 1b, and when using the O-trimethylsilyl protected compound of formula 1b is not isolated, the amount of the base is about 1.2 mole equivalents to about 5.5 mole equivalents per mole equivalent of the compound of formula 1b, more preferably about 3.5 mole equivalents to about 5.5 mole equivalents per mole equivalent of the compound of formula 1b. Most preferably, the amount of base is about 2.5 mole equivalents to about 4.5 mole equivalents per mole equivalent of the compound of formula 1b.

Further, n-pentyl haloformate is added to the suspension comprising the compound of formula 1 (“Pro-SDFC”) the base, and the solvent, providing a reaction mixture. Preferably, n-pentyl-haloformate is added in portion wise fashion. For the most part, the n-pentyl haloformate is added to the reaction mixture over a period of about 2 to about 4 hours. Preferably, it is added over a period of about 2.5 to about 3 hours. Preferably, during the addition the temperature is maintained at about 0° C. to about 35° C. More preferably, the temperature is maintained at about 20° C. to about 25° C. Preferably, the reaction mixture is maintained for about a period of about 30 minutes to about 4 hours, during this time the formation of the compound of formula 2 (“Pro-5DFCC”) is expected to occur. Preferably, the reaction mixture is maintained for about 0 hours to about 2 hours, more preferably, for about 0.5 hour to about 1 hour.

The obtained compound of formula 2 can then be converted to Capecitabine.

The conversion to Capecitabine can be done, for example, according to the process disclosed in the '949 patent or by the process disclosed herein.

Generally, such a conversion is done by a process comprising hydrolyzing the protecting groups of the compound of formula 2.

When the acylation and hydrolysis are done one-pot, an organic phase containing the intermediate 2′,3′-di-protected-5′-deoxy-5-fluoro-[N⁴-(n-pentyloxy)carbonyl]-cytidine (“Pro-5DFPCC”) of formula 2 obtained by a simple work-up can be used in the hydrolysis step without the need to isolate and recover the intermediate from the organic phase.

Preferably, the organic phase containing the intermediate 2′,3′-di-protected-5′-deoxy-5-fluoro-[N⁴-(n-pentyloxy)carbonyl]-cytidine (Pro-5DFPCC) of formula 2 is obtained by combining the reaction mixture after acylation with water to obtain a two-phase system. The phases are then separated, and the organic phase containing the compound of formula 2 is used to prepare Capecitabine. Preferably, the organic phase is an organic solution.

If required, the obtained compound of formula 2 (“Pro-5DFCC”) can also be recovered from the organic phase.

The obtained compound of formula 2 (“Pro-5DFCC”) has a purity of at least about 95% as determined by percentage area HPLC, preferably, at least 98.5% as determined by percentage area HPLC, and more preferably, a purity of at least 99% as determined by percentage area HPLC. Preferably, the content of double acylating impurity (i.e., dipentyl impurity), having the following formula:

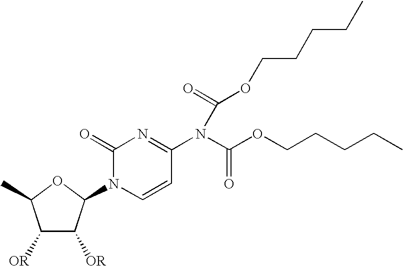

in compound of formula 2 is less then about 7% as determined by percentage area HPLC, preferably, less than about 1% as determined by percentage area HPLC, wherein R is either C(O)CH₃ or SiMe₃.

The present invention also encompasses a process for preparing Capecitabine from the compound of formula 2 (“Pro-5DFCC”) comprising removing the protecting groups of the compound of formula 2 by hydrolysis at a temperature of about −25° C. to about −5° C. to obtain Capecitabine.

The removal of the protecting groups is achieved by reacting the compound of formula 2 with a base at a temperature of about −25° C. to about −5° C., preferably, at a temperature of about −15° C. to about −5° C., i.e., basic hydrolysis of the protecting groups.

Preferably, an aqueous solution of the base, optionally containing also alcohol, preferably methanol, is reacted. If the aqueous solution doesn't contain alcohol, it is preferably further added.

Preferably, the base used in the hydrolysis step is either ammonium hydroxide or an alkali metal base. Preferably, the alkali metal base is sodium hydroxide, potassium carbonate, or sodium methylate. More preferably, the alkali metal base is sodium hydroxide

Preferably, the amount of base is about 1.0 mole equivalent to about 4.0 mole equivalents per mole equivalent of the compound of formula 2, more preferably, about 1.3 mole equivalents to about 3.0 mole equivalents per mole equivalent of starting compound of formula 2, more preferably, about 1.5 mole equivalents to about 2.5 mole equivalents per mole equivalent of starting compound of formula 2 and most preferably about 2.0 mole equivalents to about 2.5 mole equivalents per mole equivalent of starting compound of formula 2.

The compound of formula 2 (“Pro-5DFCC”) can be neat (i.e., the acylation and hydrolysis are not one pot) or in a form of an organic solution obtained from the previous step (i.e., one pot reaction). If neat, it is preferably combined with an organic solvent, thus providing a solution prior to the addition of the base. Preferably, the organic solvent is as described before.

Most preferably, the organic solvent is 2-methyl tetrahydrofuran.

Preferably, the ratio between alcohol and water in the solvent system is of about 0.5:1 v/v to about 2:1 v/v, respectively

Preferably, the ratio between the organic solvent, water and alcohol is 12:2:1 v/v, respectively.

Typically, the organic solution is cooled prior to the addition of an aqueous solution of the base.

Preferably, the cooling is to a temperature of about −5° C. to about −25° C., more preferably, to about −5° C. to about −15° C.

Typically, after the addition of the aqueous solution of the base a two-phase reaction mixture, depending on the solubility of each solvent, can be obtained. Thus, the reaction mixture can be either a one phase or two-phase reaction mixture.

Typically, the phase separation can be increased by using salted water. Preferably, the water is salted water. As used herein, the term “salted water” relates to a solution comprising water and organic or inorganic salt or mixture thereof, in concentration of about 0.5% w/w (g/g) of salt in the water to about saturation concentration. Typically, saturation can be noticed by monitoring the turbidity of the solution, i.e., the transformation of clear solution into a turbid solution.

Preferably, the organic salt is sodium acetate, potassium acetate and ammonium acetate or a mixture thereof. More preferably, the organic salt is sodium acetate.

Preferably, the inorganic salt is sodium chloride, sodium sulphate, potassium chloride, potassium sulphate, ammonium sulphate and ammonium chloride or mixture thereof, more preferably sodium chloride, barium chloride or calcium chloride.

Most preferably, the salt is sodium chloride.

The hydrolysis is performed over a period of about 20 minutes to about 3 hours. Preferably, when removing the O-acetyl group from the compound of formula 2a the hydrolysis is performed over a period of about 0.5 hour to about 3 hours, more preferably, over a period of about 1.5 hours to about 2 hours. Preferably, when removing the O-trimethylsilyl from the compound of formula 2b the hydrolysis is performed over a period of about 20 minutes to about 3 hours, more preferably, over a period of about 30 minutes to about 60 minutes.

After the hydrolysis step an acid is added to the reaction mixture. Typically, the acid addition decreases the pH to a pH where Capecitabine is more stable from further hydrolysis. Preferably, the reaction with the acid provides a pH of about 6 to about 7, more preferably, about 6.5 to about 7.

Preferably, the acid is a mineral acid, more preferably, sulfuric acid.

Typically, the acid addition is done in the presence of water, i.e. water is added to the mixture comprising Capecitabine, prior to the addition of the acid. Preferably, the water is saturated with a salt, i.e., adding brine to the mixture.

Subsequently, Capecitabine can then be recovered from the reaction mixture. The recovery can be done, for example, by separating the phases that are obtained after the addition of the acid and concentrating the organic phase to obtain a concentrate.

Optionally, prior to concentrating the organic phase, the aqueous phase is extracted.

Optionally, prior to the extraction of the aqueous phase, the organic phase obtained after the addition of the acid can be washed with water, preferably salted water, in order to remove additional impurities, such as impurity A. Preferably, the washing is done at a temperature of about 0° C. to about 40° C., more preferably 25° C. to about 35° C. Optionally the process may be repeated one or more times.

Optionally, the organic phase concentrate is re-concentrated by adding an organic solvent to the said organic phase concentrate, providing a mixture which is then concentrated again, i.e., by stripping. Preferably, the organic solvent that is used to re-concentrate the organic phase concentrate is selected from a group consisting of linear or branched ester, ketone, aliphatic hydrocarbon, aromatic hydrocarbon, ether, aliphatic nitrile derivates and mixtures thereof. Preferably, the linear or branched ester is C₂-C₆ ester, more preferably, the C₂-C₆ ester is a C₄-C₆ ester. Most preferably, the C₄-C₆ is ethyl acetate, propyl acetate, isopropyl acetate, butyl acetate, or mixtures thereof. Preferably, the ketone is C₂-C₈ ketone, more preferably, the C₂-C₈ ketone is C₃-C₈. Most preferably the C₃-C₈ is methyl iso-butyl ketone (“MIBK”), methyl ethyl ketone (“MEK”), or mixtures thereof. Preferably, the aliphatic hydrocarbon is C₅-C₈ aliphatic hydrocarbon, more preferably, the C₅-C₈ aliphatic hydrocarbon is hexane, heptane or mixtures thereof. Preferably, the aromatic hydrocarbon is C₇-C₈ aromatic hydrocarbon, more preferably, the C₇-C₈ aromatic hydrocarbon is benzene, xylene, toluene, or mixtures thereof. Preferably, the ether is C₂-C₆ ether, more preferably, the C₂-C₆ ether is C₄-C₆ and most preferably the C₄-C₆ ether is diisopropyl ether, methyl tert butyl ether, tetrahydrofuran, or mixtures thereof. Preferably, the aliphatic nitrile is C₂-C₄ aliphatic nitrile, more preferably, the C₂-C₄ aliphatic nitrile is acetonitrile, propionitrile, or mixtures thereof.

Most preferably, the organic solvent used for concentration of the organic phase is toluene.

Preferably, the stripping can be repeated several times.

After concentrating, the product is precipitated by crystallizing it. The crystallization comprises combining the concentrate with a second solvent system to provide a solution, and combining with the said solution with an anti-solvent to provide a suspension from which Capecitabine is precipitated. Preferably, the second solvent system contains any one of the above solvents, preferably acetonitrile, or a mixture of any one of the above solvent and an aromatic solvent, preferably toluene. Preferably, the second solvent system contains acetonitrile or mixture of toluene and acetonitrile

Preferably, to aid in dissolution the combination of the solvents with the concentrate can be heated. Preferably, the combination is heated to a temperature of about 30° C. to about 65° C., more preferably, it is heated to a temperature of about 35° C. to about 45° C.

Preferably, the suspension is cooled and further maintained, prior to recovering the crystalline Capecitabine.

Preferably, maintaining is at a temperature of about 35° C. to about −20°, more preferably maintaining is at a temperature of about 25 to about −5° C.

Preferably, maintaining is done for a period of about 1 hour to about 24 hours, more preferably, it is maintained for a period of about 1 hour to about 16 hours.

The precipitated Capecitabine is then filtered, washed and dried. Preferably, drying is done at a temperature of about 40° C. to about 70° C., more preferably, drying is done at a temperature of about 40° C. to about 60° C.

The obtained Capecitabine has high purity and low levels of impurities such as 2-methyl butyl or 3-methyl butyl oxycarbonyl analogues of the following formulas:

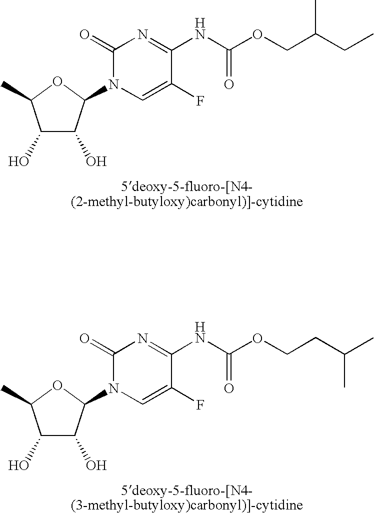

These impurities of Capecitabine are originated from the pentyl-haloformate. Thus, selecting a batch of pentyl-haloformate having a total amount of both impurities which is less than about 0.1% as determined by percentage area HPLC of the following impurities,

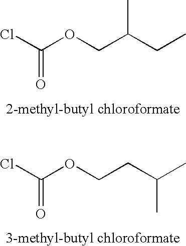

provides capecitabine having less than about 0.1% as determined by percentage area HPLC of the impurities 2-methyl butyl, 3-methyl butyl oxycarbonyl analogues or a mixture thereof.

Having thus described the invention with reference to particular preferred embodiments and illustrative examples, those in the art can appreciate modifications to the invention as described and illustrated that do not depart from the spirit and scope of the invention as disclosed in the specification. The examples are set forth to aid in understanding the invention but are not intended to, and should not be construed to, limit its scope in any way. Absent statement to the contrary, any combination of the specific embodiments described above are consistent with and encompassed by the present invention.

### EXAMPLES

### GC Method Description

GLC Conditions
Instrument: Hewlett Packard Mod. 6890 or equivalent;
Capillary Column: Fused Silica CP 30 m; i.d.=0.32 mm;
Stationary Phase: DB-1701, df=1 μm (Agilent, Part No. 123-0733);
Carrier Gas: He, 7.0 mL/minute (constant flow);
Injector mode: split (Liner: split L/P drop, glasswool Agilent P/N 5183-4647);
Split flow: 70 mL/minute;
Split ratio: 10:1;
Temperature: 60° C. initial; 10° C./min to 200° C.; 200° C. for 2 mins (for a total of 16 minutes);
Injector: 125° C.;
Detector: 250° C.;
Detector: Flame Ionization;
   H2: 30 mL/min;
   Air: 300 mL/min;
Injection Volume: 0.1 μL;
Wash Solvent: N.A.
   The retention time for pentylchloroformate is about 5 minutes.

### System Suitability Solution

Transfer about 10 mg of pentylchloroformate, accurately weighed, to a 10.0 mL volumetric flask, dissolve and made-up to volume with water.

### Test Solution

The substance to be examined.

Inject in duplicate.

### Procedure

Inject into a gas chromatography the System Suitability Solution, record the chromatogram and examine it.

The determination is not valid if:

The column efficiency for the main peak (calculated on Test Solution first preparation) is less than 15,000 theoretical plates;

Subsequently inject the Test Solution first and second preparation, record the chromatograms, examine them and measure the peak responses.

Identify the following impurities by rrt versus pentyl chloroformate:
Impurity rrt 0.862-methyl-butyl chloroformate+3-methyl-butyl chloroformate

Calculate the percentage value of related substances by automatic integration method (area percent).

Disregard any peak whose area is less than 0.04% with respect to the area of the main peak obtained in the chromatograms.

Average the two values obtained.

### HPLC Method Description

HPLC Chromatographic Condition:

| COLUMN & | Inertsil 5 μm ODS-2 250 · 4,6 mm | | |
|---|---|---|---|
| PACKING: | (P/N 5020-01102-46) | | |
| MOBILE PHASE A: | Acetic Acid 0.1%/Methanol/Acetonitrile 60/35/5 | | |
| MOBILE PHASE B: | Methanol/0.1% Acetic Acid/Acetonitrile 80/15/5 | | |
| | Time | % | % |
| | (min) | Eluent A | Eluent B |
| GRADIENT | 0 | 100 | 0 |
| | 5 | 100 | 0 |
| | 20 | 49 | 51 |
| | 25 | 15 | 85 |
| | 35 | 15 | 85 |
| | 36 | 100 | 0 |

| | |
|---|---|
| STOP TIME | 35 minutes |
| EQUILIBRIUM | 9 minutes |
| TIME: | |
| FLOW RATE: | 1.0 ml/min |
| DETECTOR: | UV at 250 nm |
| COLUMN | 40° C. |
| TEMPERATURE | |
| INJECTION | 10 μl |
| DILUENT | Water/Methanol/Acetonitrile: 60/35/5 |

### Reference Solution:

Operating in a glove-box weight about 30 mg of Capecitabine (4009AO) in a 50 ml volumetric flask and bring to volume with diluent.

### Sample Solution:

In a 50 ml volumetric flask add 0.25 ml of reaction mixture at −10° C. and bring immediately to volume with diluent.

### Procedure:

Into a high-performance liquid chromatograph equipped with a suitable injection device inject:

| | |
|---|---|
| Blank Solution (as Diluent) | for 1 time |
| Reference Solution | for 1 time |
| Sample Solution | for 1 time |

and record the chromatograms.

### Calculation:

In the chromatogram obtained calculate the residual content of 2′,3′-di-O-acetyl-5′-deoxy-5-fluoro-[N4(pentyloxy)-carbonyl)]cytidine (AcCAP, 329700) and the residual content of 2′,3′-O-carbonyl-5′-deoxy-5-fluoro-N⁴-(pentyloxycarbonyl)cytidine (Rel C, 93200H)) in area % by automatic integration.

Disregard peak at 2.28 (Pyridine), any peak whose area is less than 0.04% with respect to the area of the main peak and any peaks due to Blank Solution.

As used herein, the term “A %” refers to percent area as determined by HPLC.

As used herein, the term “Room temperature” refers to a temperature between about 20° C. and about 30° C., preferably about 25° C.

### Example 1

### Preparation of 2′,3′-di-O-acetyl-5′-deoxy-5-fluorocytidine of compound 1a (according to U.S. Pat. No. 5,472,949)

(a) From 5′-deoxy-5-fluorocytidine

5′-Deoxy-5-fluorocytidine (50 mg) was dissolved in dry pyridine (1.3 ml). To the solution was added acetic anhydride (39 ml) with stirring at 0° C. The reaction mixture was stirred for 3 hours at 0° C. After removal of the solvent under reduced pressure, the residue was partitioned between ethyl acetate and ice cooled water. The ethyl acetate layer was dried over magnesium sulfate and concentrated under reduced pressure. The residue was purified by silica gel column chromatography (dichloromethane/methanol=9/1 as an eluent) followed by recrystallization from isopropanol to give 37 mg of 2′,3′-di-O-acetyl-5′-deoxy-5-fluorocytidine: 191.5° C.-193° C., FAB-MS m/z 330 (MH⁺).

(b) From 5-fluorocytosine and 1,2,3-tri-O-acetyl-5-deoxy-.beta.-D-ribofuranose

A solution of sodium iodide (3.6 g) and chlorotrimethylsilane (794 ml) in dry acetonitrile (15 ml) was stirred with molecular sieves 4 Å (200 mg) at 0° C. for 5 minutes (colorless sodium chloride deposited during stirring). 1,2,3-Tri-O-acetyl-5-deoxy-.beta.-D-ribofuranose (2.0 g) was added and the mixture was stirred at 0° C. for 30 min. Then, a solution of the trimethylsilylated 5-fluorocytosine, freshly prepared from 5-fluorocytosine (1.12 g), in dry acetonitrile (5 ml) was added at 0° C., and stirring was continued for 3 h at room temperature. The mixture was filtered, the filtrate was concentrated in vacuum, and the residue was partitioned between dichloromethane and saturated aq. sodium bicarbonate solution. The aqueous layer was extracted with CH₂Cl₂/MeOH (10:1). The combined organic layers were dried over anhydrous sodium sulfate and evaporated under reduced pressure. The residue was purified by silica gel chromatography using CH₂Cl₂/MeOH (15:1) as an eluent, followed by recrystallization from isopropanol to give 1.24 g of 2′,3′-di-O-acetyl-5′-deoxy-5-fluorocytidine.

### Example 2

### Preparation of 5′-Deoxy-5-fluoro-[N⁴-(pentyloxy)carbonyl]-cytidine (Capecitabine)

10 g of 2′,3′-di-O-acetyl-5′deoxy-5-fluoro-cytidine were suspended in 60 ml of MeTHF, 4.2 ml of pyridine (1.7 equivalents) were added and the suspension was kept at 23-25° C. 6.9 ml (1.55 equivalents) of n-pentyl chloroformate were added portion wise during 2.5 hours, after −30′ minutes the reaction was completed, HPLC analysis showed a purity of about 98.0% with about 1.2% of dipentyl impurity. 30 ml of water were added.

The mixture was kept under stirring for 10 minutes, and then the phases were separated.

Organic phase was cooled to −17° C. and 1.6 g (1.3 equivalents) of sodium hydroxide that were dissolved in 15 ml of 1:2 water/methanol mixture were added, keeping the temperature between −20° C. and −15° C.

The reaction was completed in 30 minutes, HPLC analysis showed a purity of about 98% with a content of impurity A of about 0.5% and dipentyl impurities (of both protected and deprotected 5′deoxy-5-fluoro-cytidine) less than 0.5%.

30 ml of salted water were added and the pH was corrected to 6-7 with dilute sulphuric acid.

The phases were separated and the water phase was back-extracted with MeTHF (25 ml×2).

The combined organic phases were concentrated under vacuum at T<40° C. until 30 ml of residual volume was obtained. Then, 70 ml of toluene were added and the solution was concentrated again under vacuum until 50 ml of residual volume was obtained. Additional 50 ml of toluene were added and the solution was kept at RT for 8 hours.

The suspension was filtered and the solid was washed with toluene and dried under vacuum at 65° C.

Yield: 9.5 g equivalent to 86%.

Purity: 99.7% by HPLC.

### Example 3

### Preparation of 5′-Deoxy-5-fluoro-[N⁴-(pentyloxy)carbonyl]-cytidine (Capecitabine)

20 g of 2′,3′-di-O-acetyl-5′deoxy-5-fluoro-cytidine were suspended in 120 ml of MeTHF, 8.8 ml of pyridine (1.8 equivalents) were added and the suspension was kept at 23-25° C. 14.2 ml (1.6 equivalents) of n-pentyl chloroformate were added portion wise during 2.5 hours, after 30′ minutes the reaction was completed, HPLC analysis showed a purity of about 97.0% with about 1.9% of diacylated impurity. 60 ml of water were added.

The mixture was kept under stirring for 10 minutes, and then the phases were separated.

Organic phase was cooled to −15° C. 5 g of sodium acetate were dissolved in 12.15 g (1.5 equivalents) of sodium hydroxide 30% in water solution, and 10 ml of methanol solution were added, keeping the temperature between −15° C. and −10° C.

The reaction was completed in 2 hours, HPLC analysis shows a purity of about 97% with a content of Impurity A of about 0.5% and dipentyl impurities (sum of both protected and deprotected) less than 0.9%.

60 ml of salted water were added and the pH was corrected to 6-7 with dilute sulphuric acid.

The phases were separated and the water phase was back-extracted with MeTHF (50 ml×2).

The combined organic phases were concentrated under vacuum at T<40° C. until 60 ml of residual volume was obtained. Then, 140 ml of toluene were added and the solution was concentrated again under vacuum until 100 ml of residual volume was obtained. Additional 90 ml of toluene and 10 ml of acetonitrile were added and the solution was kept at RT for 8 hours and then at 0° C. for other 8 h.

The suspension was filtered and the solid was washed with toluene and dried under vacuum at 67° C.

Yield: 17.4 g equivalent to 80%.

Purity: 99.8% by HPLC.

### Example 4

### Preparation of 5′-Deoxy-5-fluoro-[N⁴-(pentyloxy)carbonyl]-cytidine (Capecitabine)

20 g of 2′,3′-di-O-acetyl-5′deoxy-5-fluoro-cytidine were suspended in 120 ml of MeTHF, 8.8 ml of pyridine (1.8 equivalents) were added and the suspension was kept at 23-25° C. 14.2 ml (1.6 equivalents) of n-pentyl chloroformate were added portion wise during 2.5 hours, after 30′ minutes the reaction was completed. HPLC analysis showed a purity of about 97.0% with about 1.9% of dipentyl impurities. 60 ml of water were added.

The mixture was kept under stirring for 10 minutes, and then the phases were separated.

Organic phase was cooled to −15° C. 3.0 g of sodium chloride were dissolved in 17.0 g (2.1 equivalents) of sodium hydroxide 30% water solution, 10 ml of water and 10 ml of methanol; this solution was added, keeping the temperature between −5° C. and −10° C.

The reaction was completed in 2 hours. HPLC analysis showed a purity of about 98% with a content of impurity A of about 1.5% and dipentyl impurities (sum of both protected and deprotected) less than 0.9%.

60 ml of salted water were added and, keeping the temperature less than −5° C., the pH was corrected to 6-7 with dilute sulphuric acid.

The mixture was warmed at 25-30° C. and the phases were separated: extraction with salted water was repeated until the content of impurity A in organic phase was less than 0.4%. After this organic phase was washed with 30 ml of water.

All the water phases were collected and were back-extracted with MeTHF (50 ml×2).

The combined organic phases were filtered and concentrated under vacuum at T<40° C. until 60 ml of residual volume was obtained. Then, 140 ml of toluene were added and the solution was concentrated again under vacuum until 60 ml of residual volume was obtained. Additional 140 ml of toluene were added and the solution was concentrated again under vacuum until 100 ml of residual volume was obtained. 20 ml of acetonitrile are added and the temperature was raised to 45° C. until dissolution of eventual precipitate. Additional 140 ml of toluene were added and the solution was cooled to 0° C. and was kept at this temperature for 16 hours.

The suspension was filtered and the solid was washed with toluene and dried under vacuum at 70° C.

Yield: 17.4 g equivalent to 80%.

Purity: 99.90% by HPLC.

### Example 5

### Preparation of 5′-Deoxy-5-fluoro-[N⁴-(pentyloxy)carbonyl]-cytidine

A batch of n-pentyl chloroformate having pentyl chloroformate isomers content of 0.1% as measured by GC was used in the reaction as described in example 2 to prepare capecitabine.

Yield: 8.5 g equivalent to 78%.

Isomers impurity content: 0.1% (A %) by HPLC.

### Example 6

### Preparation of 5′-Deoxy-5-fluoro-[N⁴-(pentyloxy)carbonyl]-cytidine

A batch of n-pentyl chloroformate having pentyl chloroformate isomers content of less then about 0.04% as measured by GC was used in the reaction as described in example 2.

Yield: 15.4 g equivalent to 71%.

Isomers impurity content: <0.04% (A %) by HPLC.

### Example 8

### Preparation of 5′-Deoxy-5-fluorocytidine from 2′,3′-di-O-acetyl-5′deoxy-5-fluorocytidine

2′,3′-di-O-acetyl-5′deoxy-5-fluorocytidine (30 g) was dissolved in 250 ml of methanol, the solution was cooled to 0-5° C. and 1.8 g of sodium methoxide 30% solution in methanol (0.1 equivalents) was added.

The hydrolysis was complete in 45 minutes, and then the mixture was neutralized with hydrochloric acid.

The solution was concentrated under vacuum at a temperature below 50° C. until oil residue. 40 ml of pyridine were added and the concentration was continued again until oil residue.

### Example 9

### Preparation of 5′-Deoxy-5-fluorocytidine from 2′,3′-di-O-acetyl-5′deoxy-5-fluorocytidine

30.0 g of 2′,3′-di-O-acetyl-5′deoxy-5-fluorocytidine were dissolved in 180 ml of methanol, 10.5 ml of 25% ammonia in water solution were added and the solution was heated to a temperature of about 30° C.-40° C. for a period of about 2-3 hours. The solution was concentrated under vacuum at 40° C. until 60 ml, then 100 ml of THF were added and the mixture was distilled until 60 ml at atmospheric pressure (in these conditions the azeotrope THF/methanol has a boiling point of 60° C.).

Other 100 ml of THF were added and distillation was repeated until 60 ml, in this stage a suspension was obtained. At the end the suspension was diluted with THF until 90 ml and cooled to 0° C. for 3 hours.

The solid as filtered and dried in vacuum at 60° C. 12 hours.

Yield: 19.5 g equivalent to 88% mol/mol.

Purity: 99.90% (A %) by HPLC

### Example 10

### Preparation of 5′-Deoxy-5-fluorocytidine from 2′,3′-di-O-acetyl-5′deoxy-5-fluorocytidine

30.0 g of 2′,3′-di-O-acetyl-5′deoxy-5-fluorocytidine were dissolved in 180 ml of methanol, 10.5 ml of 25% ammonia in water solution were added and the solution was heated at 45-55° C. for 2-3 hours.

The solution was concentrated at atmospheric pressure until 90 ml, then 210 ml of toluene were added and the mixture was distilled at atmospheric pressure until internal temperature of 75-85° C. (in these conditions the azeotrope toluene/methanol has a boiling point of 63.8° C.). 30 ml of acetonitrile were added and the mixture was stirred at 70-80° C. for 30 minutes, then 60 ml of toluene were added.

At the end the suspension was cooled to 5° C. for 3 hours.

The solid was filtered and dried under vacuum at 60° C. for 12 hours.

Yield: 21.0 g equivalent to 95% mol/mol.

Purity: 99.70% (A %) by HPLC.

### Example 11

### Preparation of 5′-Deoxy-5-fluoro-[N⁴-(pentyloxy)carbonyl]-cytidine

30 g of 5′deoxy-5-fluoro-cytidine from the preceding step was dissolved in 90 g of pyridine (3 volumes) and 90 ml of dichloromethane (3 volumes), the solution was cooled to 0-5° C. and chlorotrimethylsilane (3 equivalents, 30 g) was added and the temperature was left to rise until RT and kept for 30 minutes, 200 ml of dichloromethane (about 7 volumes) were added the suspension was cooled to −15/-10° C. 30 g of n-pentyl chloroformate (2.1 equivalents) were added and the temperature rose until 0-5° C. and kept for 2 hours. 300 ml of water (10 volumes) were added and, keeping the temperature below 5° C., the mixture was acidified with dilute sulphuric acid until pH 1.0-1.5. Phases were separated and the water phase was discarded.

Organic phase was cooled to −10/−15° C. and 30 g of sodium hydroxide 32% in water (2,6 equivalents) dissolved in 60 ml of methanol were added, keeping the temperature between −15° C. and −10° C. After 30 minutes, the reaction was completed and 210 ml of salted water was added and the pH was corrected to 6-7 with dilute sulphuric acid.

Phases were separated and the water one was back-extracted with dichloromethane (75 ml×2).

All the organic phases were combined and were concentrated under vacuum at a temperature of less than about 40° C. until 30 ml of residual volume. 210 ml of toluene were added and the solution was concentrated again under vacuum until 150 ml of residual volume, other 150 ml of toluene were added and the solution was kept at room temperature for 8 hours.

The suspension was filtered and the solid washed with toluene and was dried under vacuum at 50° C.

Yield: 36.6 g equivalent to 82% from 2′,3′-di-O-acetyl-5′deoxy-5-fluorocytidine.

### Example 12

### Preparation of 5′-Deoxy-5-fluoro-[N⁴-(pentyloxy)carbonyl]-cytidine

20 g of 5 ′deoxy-5-fluoro-cytidine were suspended in 140 ml of 2-methyl-tetrahydrofuran, 32.6 ml of pyridine (4.9 equivalents) were added and the suspension was cooled to 15° C. 31.3 ml of chlorotrimethylsilane (3.1 equivalents) during 1 hour, then the temperature was left to rise until 25° C. and kept for 60 minutes. 19.2 ml of n-pentyl chloroformate (1.6 equivalents) were added during 30 minutes, then the reaction mixture was left under stirring for 1.5 hours. At the end, 60 ml of water (3 volumes) were added, the mixture was stirred for 15 minutes and then phases were separated.

Organic phase was cooled to −10/−5° C. and 27.5 g of sodium hydroxide 30% in water (2,5 mol/mol, 1.25 equivalents) diluted with 15.5 ml of methanol were added while keeping the temperature below −5° C. After 90 minutes, the reaction was completed and 120 ml of water were added. pH was corrected to 6-7.5 with dilute sulphuric acid while keeping the temperature below 5° C., then the mixture was warmed to 25° C. and the phases were separated.

Organic phase was washed with a 20% solution of sodium chloride in water to reduce the content of 5′deoxy-5-fluoro-cytidine.

At the end, organic phase was washed with 35 ml of water.

All water phases were combined and back-extracted with 2-methyl-tetrahydrofuran (50 ml×2).

All the organic phases were combined and were concentrated under vacuum at a temperature of less than about 45° C. until 50 ml of residual volume. 175 ml of toluene were added and the solution was concentrated again under vacuum at a temperature of less than about 45° C. until 50 ml of residual volume, other 175 ml of toluene were added and the distillation was continued until 125 ml. During this step, Capecitabine precipitated. 25 ml of acetonitrile were added and the suspension was stirred at 40-45° C. until dissolution, then 125 ml of toluene were added and the mixture was cooled at 0° C. for 3 hours.

The suspension was filtered and the solid washed with toluene and dried under vacuum at 65° C.

Yield: 23.55 g equivalent to 79.6% from 5′deoxy-5-fluorocytidine.

Purity: 99.85% (A %) by HPLC.

### Example 13

### Preparation of 5′-Deoxy-5-fluoro-[N⁴-(pentyloxy)carbonyl]-cytidine

In a 250 ml reactor 14 ml (1 volume) of 2-methyl-tetrahydrofuran, 19.1 ml of pyridine and 14 g of 5′-Deoxy-5-fluorocytidine were loaded and stirred under nitrogen at 35-50° C. obtaining a suspension. 17.5 ml of chlorotrimethylsilane were added by a syringe during 60 min: the product dissolved, then pyridinium salts precipitate off. At the end of the addition a suspension was obtained and the reaction mixture was left under stirring at 35-50° C. for 1 hour and completion was checked by HPLC, then the suspension was cooled, diluted with 71 ml (6 volumes) of 2-methyl-tetrahydrofuran and cooled under nitrogen at 25-30° C. 10.9 ml of n-pentyl chloroformate were added dropwise during 15 min. The mixture was left under good stirring for 1.0 hour.

Completion of the reaction was checked by HPLC and then 56 ml of water were added and the mixture stirred for 10 min. Phases were separated and the organic one was cooled to a temperature of less than about −5° C. A 30% solution of NaOH in water (12.7 ml) and then 10 ml of methanol were added and the mixture was stirred for 1.0 hours. Completion of the reaction was checked by HPLC, 56 ml of water were added and the mixture neutralized to pH 6-7.5 with diluted sulfuric acid (50% aq), the whole operation keeping the temperature below 5° C.

Temperature was raised to 25-30° C. and phases were separated: organic phase was washed with 40 ml of water salted with 8.0 g of NaCl and then with 10 ml of demineralised water.

Organic solution was concentrated under vacuum at a temperature of less than about 45° C. until 3-4 volumes residual.

The solution was cooled to RT and filtered through a dicalite pad to remove traces of salts.

To the 2MeTHF solution from the previous step, 132 ml of toluene (9.4 volumes) were added and concentration was carried on at a temperature of less than about 45° C. until 4 residual volumes (56 ml).

Other 132 ml of toluene were added and concentration continued at the same temperature until 6.7 residual volumes. During this operation the product precipitated off.

19 ml of acetonitrile (1.35 v/w) were added and the mixture was warmed at 40-45° C. and stirred until dissolution, then 114 ml (8.1 volumes) of toluene were added and the mixture cooled at 0±5° C. and kept at this temperature for at least 1 hour, then filtered.

The solid was washed two times with toluene, then vacuum dried at 40-65° C. for not less then 4 hours.

Yield: 16.4 g-78.7% mol.

Purity>99.50% (A %) by HPLC

### Example 14

### Comparative Example: Preparation of Capecitabine Effect of Temperature on the Hydrolysis

15 g of 2′,3′-di-O-acetyl-5′deoxy-5-fluoro-cytidine (Ac-5DFC), 90 ml of 2-methyl-tetrahydrofuran and 5.9 ml of pyridine were charged in a reactor and the mixture was stirred under nitrogen at a temperature of about 25° C.±5° C. to obtain a suspension. 9.3 ml of n-pentyl chloroformate were added drop wise for a period of at least 2 h. After the addition, the mixture was left under good stirring for 60 min.

Completion of the reaction was checked by HPLC (Ac-5DFC≦0.3% HPLC area percent) and then 45 ml of water were added and the mixture was stirred for 15 min. Phases were separated and the organic one was kept for the next step.

70 ml of organic layer coming from acylation step (corresponding to 12.0 g of Ac-5DFC) was stirred at a temperature of about −10° C.±3° C. NaOH 30% solution corresponding to 2.1 mol/molsub was mixed with 1.8 g of NaCl and 6.0 ml of water was slowly added, followed by addition of 6.0 ml of methanol: a biphasic mixture was obtained.

Reaction progress was monitored by HPLC. After 1.5 hour the reaction was completed

Purity: Capecitabine: 99.3% (A %) by HPLC, with formation of small amount of impurities, related impurity A: 0.3% (A %) by HPLC.

### Example 15

### Comparative Example: Preparation of Capecitabine Effect of Temperature on the Hydrolysis

35 ml of organic layer coming from acylation step of example 14 (corresponding to 5.0 g of Ac-5DFC) was stirred at 25±5° C., NaOH 30% solution corresponding to 2.1 mol_{NaOH}/mol_{sub} was mixed with 0.8 g of NaCl and 2.5 ml of water were slowly added, followed by 2.5 ml of methanol: a biphasic mixture was obtained.

Reaction progress was monitored by HPLC. After 2 hours reaction was completed

Purity: Capecitabine 76.9% (A %) by HPLC, impurities (mainly related impurity A) 20.6% (A %) by HPLC.

### Example 16

### Comparative Example: Preparation of Capecitabine Effect of Temperature on the Hydrolysis

35 ml of organic layer coming from acylation step of example 14 (corresponding to 5.0 g of Ac-5DFC) was cooled at −10±3° C., NaOH 30% solution corresponding to 2.1 mol_{NaOH}/mol_{sub} was slowly added, followed by 4 ml of methanol: a homogenous solution was obtained.

Reaction progress was monitored by HPLC. After 2 hours reaction was completed.

Purity: Capecitabine 98.6% (A %) by HPLC with formation of about 0.9% of related impurity A.

### Example 17

### Comparative Example: Preparation of Capecitabine Effect of Temperature on the Hydrolysis

35 ml of organic layer coming from acylation step of example 14 (corresponding to 5.0 g of Ac-5DFC) was stirred at 25+5° C., NaOH 30% solution corresponding to 2.1 mol_{NaOH}/mol_{sub} was slowly added, followed by 3.0 ml of methanol: a homogenous solution was obtained

Reaction progress was monitored by HPLC. After 2 hours reaction was completed.

Purity: Capecitabine 71.8% (A %) by HPLC, with formation of relevant amount of impurities (mainly related impurity A 23.1%)

### Example 18

### Comparative Example: Preparation of Capecitabine Effect of Temperature on the Hydrolysis

An organic phase obtained from the acylation step containing ⅙ (w/v) 2′,3′-di-O-acetyl-5′-deoxy-5-fluoro-[N⁴-(n-pentyloxy)carbonyl]-cytidine (AcCAP) was cooled to −15° C. 1.5 mol_{NaOH}/mol_{sub} of sodium hydroxide as 30% water solution was mixed with a sodium chloride solution (0.18 w/w of NaCl diluted in 0.5 v/w of water) and then were added followed by adding 0.5 v/w of methanol, and the biphasic mixture was stirred. After 1 hour 97.92% (A %) of Capecitabine was detected and un-reacted Capecitabine intermediate (AcCAP) was 0.28% (A %), related A impurity was about 0.72% (A %). The reaction mixture was warmed to 25° C., the mixture remained biphasic; after 30 minutes the Capecitabine content was 88.37% (A %) and related impurity A was about 9.78% (A %).

We claim:

1. A process for preparing Capecitabine of the following formula: 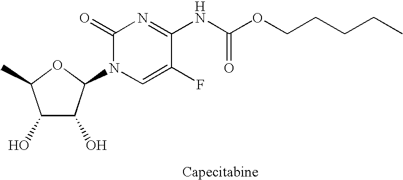
comprising: a) reacting the 2′,3′-di-protected-5′deoxy-5-fluorocytidine of formula 1: 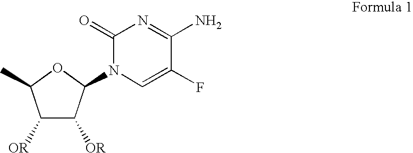
and about 1.1 mole equivalents to about 3.0 mole equivalents of pentyl-haloformate per mole equivalent of the compound of formula 1, wherein R is either C(O)CH₃ or SiMe₃, and about 1.5 mole equivalents to about 3.2 mole equivalents of a base per mole equivalent of the compound of formula 1 to obtain 2′,3′-di-protected-5′-deoxy-5-fluoro-[N⁴-(n-pentyloxy)carbonyl]-cytidine of formula 2: 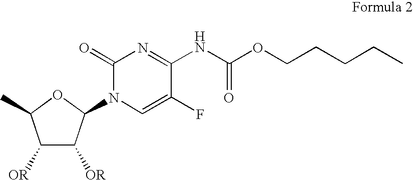
b) removing the protecting groups by hydrolysis at a temperature of about −5° C. to about −25° C. to obtain Capecitabine salt, wherein R is H; and c) adding an acid to obtain Capecitabine.

2. The process of claim 1 wherein the pentyl-haloformate is either chloroformate or bromoformate.

3. The process of claim 2 wherein the pentyl-haloformate is chloroformate.

4. The process of claim 1, wherein when R is C(O)CH₃, the amount of pentyl-haloformate is about 1.35 mole equivalents to about 2.0 mole equivalents per mole equivalent of the compound of formula 1; or wherein when R is SiMe₃, the amount of pentyl-haloformate is about 1.1 mole equivalents to about 3.0 mole equivalents per mole equivalent of the compound of formula 1.

5. The process of claim 1, wherein the base in step (a) is either an organic base or inorganic base.

6. The process of claims 5, wherein the organic base is selected from a group consisting of: pyridine, triethylamine (“TEA”), N,N-diisopropylethylamine(“DIPEA”), N-methyl-morpholine, imidazole, dimethylaminopyridine(“DMAP”), and mixtures thereof.

7. The process of claim 6, wherein the organic base is pyridine.

8. The process of claims 5, wherein the inorganic base is an alkali metal base or ammonium hydroxide.

9. The process of claims 8, wherein the alkali metal base is selected from a group consisting of: sodium carbonate, potassium carbonate, sodium hydrogencarbonate, potassium hydrogencarbonate, magnesium oxide, and mixtures thereof.

10. The process of claims 9, wherein the alkali metal base is potassium carbonate.

11. The process of claim 1, wherein when R is C(O)CH₃, the amount of the base in step (a) is about 1.7 mole equivalents to about 2.2 mole equivalents per mole equivalent of the compound of formula 1; or wherein when R is SiMe₃, the amount of the base in step (a) is 1.5 mole equivalents to about 3.2 mole equivalents per mole equivalent of the compound of formula 1.

12. The process of claim 1, wherein the reaction in steps (a) and (b) further comprise the presence of a single solvent or a mixture of solvents.

13. The process of claim 12, wherein the single solvent is selected from a group consisting of: chlorinated aliphatic hydrocarbon, ketone, ester, and ether.

14. The process of claim 13, wherein the single solvent is selected from a group consisting of: C₁₋₄ chlorinated aliphatic hydrocarbon, C₃-C₆ ketone, C₄-C₆ ester, and C₂-C₆ ether.

15. The process of claim 14, wherein the single solvent is selected from a group consisting of: dichloromethane, methyl-ethyl ketone (“MEK”), methyl-isobutyl ketone (MIBK), a mixture of MEK and MIKB, ethyl acetate, isopropyl acetate, and 2-methyl-tetrahydrofuran (“2-MeTHF”).

16. The process of claim 12, wherein the mixture of solvents contains 2-methyl-tetrahydrofuran (“2-MeTHF”) and a solvent selected from a group consisting of: dichloromethane, methyl-ethyl ketone (“MEK”), methyl-isobutyl ketone (MIBK), a mixture of MEK and MIKB, ethyl acetate, isopropyl acetate, and a mixture thereof.

17. The process of claim 1, wherein 2′,3′-di-protected-5′-deoxy-5-fluoro-[N⁴-(n-pentyloxy)carbonyl]-cytidine of formula 2 is not isolated prior to step (b).

18. The process of claim 1, wherein the removal of the protecting groups is achieved by reacting the compound of formula 2 with a base at a temperature of about −25° C. to about −5° C.

19. The process of claim 18, wherein the temperature is about −15° C. to about −5° C.

20. The process of claim 18, wherein the base is either ammonium hydroxide or an alkali metal base.

21. The process of claim 20, wherein the alkali metal base is sodium hydroxide, potassium carbonate, or sodium methylate.

22. The process of claim 21, wherein the alkali metal base is sodium hydroxide.

23. The process of claim 18, wherein the amount of base is about 1.0 mole equivalent to about 4.0 mole equivalents per mole equivalent of the compound of formula 2.

24. The process of claim 18, wherein an aqueous solution of the base is reacted.

25. The process of claim 24, wherein the aqueous solution comprises a mixture of alcohol and water.

26. The process of claim 25, wherein the alcohol is methanol.

27. The process of claim 25, wherein the water is salted water.

28. The process of claim 27, wherein the salt is sodium chloride.

29. The process of claim 18, wherein the hydrolysis is a bi-phasic reaction.

30. The process of claim 1, further comprising recovering capecitabine.

31. A process for preparing capecitabine from 2′,3′-di-protected-5′-deoxy-5-fluoro-[N4-(n-pentyloxy)carbonyl]-cytidine of formula 2: 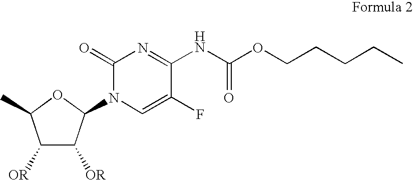
comprising removing the ester groups of Formula 2 by hydrolysis at a temperature of about −5° C. to about −25° C. to obtain Capecitabine salt; and adding an acid to obtain Capecitabine.

32. A process for preparing 2′,3′-di-protected-5′-deoxy-5-fluoro-[N4-(n-pentyloxy)carbonyl]-cytidine of formula 2: 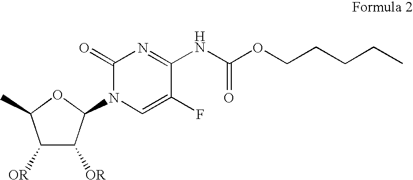
comprising reacting 2′,3′-di-protected-5′deoxy-5-fluorocytidine of formula 1: 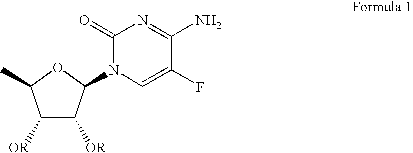
and about 1.1 mole equivalents to about 3.0 mole equivalents of pentyl-haloformate per mole equivalent of the compound of formula 1 and about 1.5 mole equivalents to about 3.2 mole equivalents of a base per mole equivalent of the compound of formula 1, wherein R is either C(O)CH₃ or SiMe₃.

33. A process for preparing Capecitabine comprising preparing 2′,3′-di-protected-5′-deoxy-5-fluoro-[N4-(n-pentyloxy)carbonyl]-cytidine of formula 2 according to the process of claim 32 and converting it to Capecitabine.
